(19) United States
(12) Patent Application Publication
(10) Pub. No.: US 2008/0254071 A1
(43) Pub. Date: Oct. 16, 2008

(54) Homeopathic composition for alleviating allergy symptoms

(75) Inventors: Erin Taraborrelli, Oakville (CA); Marvin Heuer, Oakville (CA); Ken Clement, Oakville (CA); Megan Thomas, Oakville (CA); Shan Chaudhuri, Brampton (CA)

Correspondence Address: IOVATE HEALTH SCIENCE RESEARCH INC., 381 North Service Road West, Oakville, ON, L6M 0H4, CA

(73) Assignee: Iomedix Allergy International SRL, Belleville (BB)

(21) Appl. No.: 12/099,920

(22) Filed: Apr. 9, 2008

Related U.S. Application Data
(60) Provisional application No. 60/910,997, filed on Apr. 10, 2007.

(51) Int. Cl. A61K 39/35; A61P 37/08

(52) U.S. Cl. 424/275.1

(57) ABSTRACT

The present invention is directed towards a homeopathically designed composition comprising effective amounts of Thussilago petasites (butterbur) and dilute amounts of Ambrosia artemisiifolia for the alleviation of symptoms of an allergy. According to various aspects, the present invention may further comprise combinations of Allium cepa, Echineacea angustifolia, Euphrasia officinalis (eyebright), Pulsatilla nigricans, Pothos foetidus, Ignatia amara (St Ignatius' bean), and Thuja occidentalis (Tree of Life) to synergistically and additionally alleviate the symptoms of an allergy.

### RELATED APPLICATIONS

The present application is related to and claims benefit of priority to U.S. Provisional Patent Application Ser. No. 60/910,997, entitled “Homeopathic Composition for Alleviating Allergy Symptoms,” filed Apr. 10, 2007, the disclosure of which is hereby fully incorporated by reference.

### FIELD OF THE INVENTION

The present invention is related to homeopathic compositions and methods for the alleviation of allergy symptoms.

### BACKGROUND OF THE INVENTION

Immunity is a result of the adaptive immune response of mammals. In the case of an adaptive immune response, the body has a “memory” for previously encountered antigens such that a second, more vigorous response is mounted by the body upon a second exposure to the same antigen.

In conventional medicine, the normal functioning of the adaptive immune response of mammals is taken advantage of in that vaccines are routinely administered for diseases such as measles, rubella, influenza, and recently cervical cancer. Specifically, denatured antigens and substances known to active the innate immune system, adjuvants, are administered to a mammal. The denatured antigens are essentially harmless to the mammal, yet activate the adaptive immune system to produce antibodies, or immunogens, which can neutralize proteins similar to that which the denatured antigens given. After this, when the body encounters a virus or other pathogen having a similar make-up, the body can quickly produce antibodies against the invader due to the adaptive immune system.

This system of “vaccination” is also employed in allergy immunotherapy, wherein a treatment consisting of small doses of allergens is injected into a mammal, resulting in the alleviation of allergy symptoms as a result of an immune response. This method of treatment of is particularly useful in case of treating an individual against inhaled allergen, and anaphylaxis.

Since the antigens, or proteins, which are administered to an individual, elicit an immune response in the body to confer future immunity against the specific antigen, they are referred to as therapeutic proteins. This concept is the basis for homeopathic medicine. A substance which elicits a very similar symptomatic response is administered to an individual at a low dose; enough to active the adaptive immune system. For example, if the symptoms that a patient is expressing are similar to the symptoms expressed by a patient upon exposure to a particular pathogen, then the same pathogen is used a very low doses to treat the patient. The pathogen upon administration induces the body to produce immunogens which combat the symptoms expressed, and thus providing a remedy.

In homeopathic medicine, the pathogen is generally administered in an aqueous solution at a very low dose. Traditionally, amounts of substances present in a homeopathic remedy are often referred to as its dilution factor wherein a 1:10 dilution is equal to 1× (× potencies). For example, a 10× dilution is a dilution factor of 1010. Therefore the actual amount of antigen administered is very small. As such, it is understood that in the dilution, an essential property of the substance that was diluted is retained in the aqueous medium; enough to elicit a response of the adaptive immune system in a manner similar to a conventional vaccine.

Remedies given in homeopathic medicine are generally given as oral compositions. They are often placed on or under the tongue. By administering in this way, then antigenic proteins are not destroyed by gastric and intestinal juices, thus allowing them to reach lymphoid tissues intact. Generally, homeopathic treatments are administered orally, sublingually, nasally, ophthalmically and topically. In these routes of administration, chemical degradation of the antigen is most likely avoided.

### SUMMARY OF THE INVENTION

The present invention is directed towards a homeopathically designed composition comprising effective amounts of Thussilago petasites (butterbur) and dilute amounts Ambrosia artemisiifolia for the alleviation of symptoms of an allergy. In an aspect of the present invention, the Ambrosia artemisiifolia is present in dilute amounts able to induce the body of a mammal to produce immunogens able to alleviate the symptoms of an allergy. In a further aspect of the invention, the composition may be administered to the mammal orally.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

As used herein the term ‘allergy’ is defined as being an abnormal hypersensitivity to substances which are normally harmless that produces a bodily response in a mammal with symptoms such as runny nose, spasms of sneezing, itchy eyes, watery eyes, itchy rash, and eczema.

The present invention is directed towards a homeopathic composition, for the alleviation of symptoms of an allergy, comprising effective amounts of Thussilago petasites (butterbur) and dilute amounts of Ambrosia artemisiifolia. An aspect of the present invention comprises at least an acceptable amount of Thussilago petasites (butterbur) and a homeopathically acceptable amount of Ambrosia artemisiifolia. According to various aspects, the present invention may further comprise combinations of Allium cepa, Echineacea angustifolia, Euphrasia officinalis (eyebright), Pulsatilla nigricans, Pothos foetidus, Ignatia amara (St Ignatius' bean), and Thuja occidentalis (Tree of Life).

Tussilago Petasites (Butterbur)

Butterbur (Tussilago petasites) is a perennial shrub found throughout Europe and parts of North America and Asia. Butterbur has been clinically shown to be as effective as a common antihistamine, cetirizine, for treating seasonal allergic rhinitis. Asthma has also been effectively treated with Butterbur extract. Butterbur is thought to act in treating inflammation-related conditions, such as allergies, by inhibiting the biosynthesis of leukotrienes by one of the main active constituents of Butterbur, petasine.

Leukotrienes are secreted lipid mediators of the inflammation response produced by leukocytes from arachidonic acid, which is a precursor for several mediators involved in inflammation. Production of leukotrienes is increased during inflammation and can produce and sustain the symptoms of inflammation.

It is herein understood by the inventors that an inhibitor of leukotriene biosynthesis, such as butterbur, will effectively reduce inflammation observed in allergic responses, resulting in a decrease in the severity of allergy related symptoms.

In an embodiment of the present invention, which is set forth in greater detail in the examples below, the homeopathic composition comprises Tussilago petasites (butterbur). A serving of the homeopathic composition comprises from about 10 mg to about 50 mg of Tussilago petasites (butterbur). The preferred dosage of a serving of the homeopathic composition comprises about 24 mg of Tussilago petasites (butterbur).

Allium Cepa

Allium cepa, most commonly referred to as the onion, is an edible plant that has been used worldwide predominately for culinary purposes. However, the onion has also been used in traditional medicine to treat boils, wounds and stings, to relieve coughs, bronchitis, asthma, some gastro-intestinal disorders and headaches. Onions are now recognized as a significant source of anti-inflammatory and antioxidant components, such as quercetin.

Quercetin is flavonoid that has demonstrated significant anti-inflammatory activity as a result of direct inhibition of several initial processes of inflammation. Quercetin has been shown to suppress several proinflammatory and allergy signaling molecules in human mast cells, which likely contributes to alleviating allergy symptoms.

More relevant however, is the fact that Allium cepa has long been shown to cause sneezing and itchy red eyes when administered in large doses. However, in homeopathic medicine, substances known to elicit adverse effects in large does are administered in highly dilute forms to induce the body to produce immunogens able to alleviate the symptoms of an allergy. Therefore, it is herein understood by the inventors that a highly dilute form of Allium cepa will facilitate the alleviation of allergy symptoms, mainly sneezing and itchy red eyes.

In an embodiment of the present invention, which is set forth in greater detail in the examples below, the homeopathic composition comprises Allium cepa. A serving of the homeopathic composition comprises from about 4× to about 8× of Allium cepa. The preferred dosage of a serving of the homeopathic composition comprises about 6× of Allium cepa.

Ambrosia Artemisiifolia

Ambrosia artemisiifolia, is a widespread plant in North America and is most commonly known as ragweed. Subcutaneous administration of a standardized extract of Ambrosia pollen has been shown to reduce the number of days with rhinitus symptoms as well as attenuate asthmatic symptoms. Thus, this injective immunotherapy was determined to be safe and effective for alleviation and attentuation of allergy symptoms.

A compilation of reports of homeopathic providings based upon observed effects of the homeopathic remedies on volunteers has shown Ambrosia to be a potent remedy for hay-fever. The administration of high amounts of Ambrosia resulted in the symptoms of hay-fever. Administration of diluted forms of Ambrosia promotes the immune system to become less symptomatically reactive to allergens, resulting in alleviation of the symptoms of an allergy.

In an embodiment of the present invention, which is set forth in greater detail in the examples below, the homeopathic composition comprises Ambrosia artemisiifolia. A serving of the homeopathic composition comprises from about 4× to about 8× of Ambrosia artemisiifolia. The preferred dosage of a serving of the homeopathic composition comprises about 6× of Ambrosia artemisiifolia.

Echinacea Angustifolia

Echineacea, commonly called Echinacea, refers to a genus consisting of nine species of flowering plants, which are all native to eastern North America. All species of echinacea possess compound phenols, which are often purported to be responsible for echinacea's health benefits. Most notably of theses health benefits is the ability to boost the body's immune system and ward off infections, particularly the common cold.

In homeopathic medicine, echinacea is often used help the immune system to overcome infection as well as to alleviate lymphatic inflammation. Lymphatic inflammation inhibits the ability of the body to eliminate allergens leading to an extended period of allergic response. By decreasing lymphatic inflammation the body's immune system is better able to eliminate allergens and attenuate periods of allergic response.

In an embodiment of the present invention, which is set forth in greater detail in the examples below, the homeopathic composition comprises Echinacea angustifolia. A serving of the homeopathic composition comprises from about 1× to about 4× of Echinacea angustifolia. The preferred dosage of a serving of the homeopathic composition comprises about 1× of Echinacea angustifolia.

Euphrasia Officinalis

Euphrasia, commonly called eyebright, is a flowering plant that is found throughout the world. As the common name implies this plant was often used in the treatment of various eye conditions. The herb is commonly used as a homeopathic remedy to relieve inflammation caused by colds, sinus infections and sore throats, and is known to ameliorate burning, watery eyes. Sinus infections and watery eyes are quite often present in situations of allergic responses and so alleviation of these by use of a homeopathic remedy containing Euphrasia, would reduce the symptoms of an allergy.

In an embodiment of the present invention, which is set forth in greater detail in the examples below, the homeopathic composition comprises Euphrasia officinalis. A serving of the homeopathic composition comprises from about 4× to about 8× of Euphrasia officinalis. The preferred dosage of a serving of the homeopathic composition comprises about 6× of Euphrasia officinalis.

Pulsatilla nigricans

Pulsatilla, is referred to commonly as the pasque flower, and is a deciduous perennial flower found in North America. Pulsatilla nigricans is commonly administered, by practitioners of homeopathic remedies, to patients suffering from burning, itchy eyes. By using highly dilute forms of Pulsatilla, it is understood that the body of a mammal will produce immunogens which are able to alleviate the symptoms of an allergy.

In an embodiment of the present invention, which is set forth in greater detail in the examples below, the homeopathic composition comprises Pulsatilla nigricans. A serving of the homeopathic composition comprises from about 4× to about 8× of Pulsatilla nigricans. The preferred dosage of a serving of the homeopathic composition comprises about 6× of Pulsatilla nigricans.

Pothos Foetidus

Pothos foetidus, commonly referred to as skunk cabbage, is a low growing, foul smelling plant that prefers wetlands, and is commonly found in eastern North America and northeastern Asia. Skunk cabbage has shown a potent ability to treat individuals suffering from asthma, and to reduce periods of excessive sneezing. Pothos foetidus has shown the ability to eliminate symptoms in many cases of situational asthma. For example, a woman who was always wheezing while dusting was directed to take Pothos foetidus and is now able to dust her entire house without suffering from disruptive wheezing.

In an embodiment of the present invention, which is set forth in greater detail in the examples below, the homeopathic composition comprises Pothos foetidus. A serving of the homeopathic composition comprises from about 4× to about 8× of Pothos foetidus. The preferred dosage of a serving of the homeopathic composition comprises about 6× of Pothos foetidus.

Ignatia Amara

Ignatia amara, St. Ignatius' bean, is a homeopathic remedy that is derived from the bean of a small tree that is endogenous to the Philippine Islands and China. This herb has been used as a homeopathic remedy for a magnitude of problems, such as headaches, sore throat, trembling, nervousness, insomnia, heart palpitations, gas, indigestion, and tickling cough. For allergy relief it will act as a remedy for sinus headaches, aiding to relieve the constant pain above the bridge of the nose, as well as acting to reduce tickling coughs that may result from irritation.

In an embodiment of the present invention, which is set forth in greater detail in the example below, the homeopathic composition comprises Ignatia amara. A serving of the homeopathic composition comprises from about 4× to about 8× of Ignatia amara. The preferred dosage of a serving of the homeopathic composition comprises about 6× of Ignatia amara.

Thuja Occidentalis

Thuja occidentalis is an evergreen coniferous tree that is native to the northeastern United States and southeastern Canada. This herb was first used by the native Indians of Canada for the treatment of weakness from scurvy. In folk medicine Thuja is commonly used as a remedy for acute and chronic infections of the upper respiratory tract.

Thuja was shown to lead to a dose-dependent shift in white cell blood counts within 4 hours, when administered to mice. Additionally, treatment of mice with Thuja, also showed the ability to significantly increase levels of pro-inflammatory cytokines such as tumor necrosis factor, in comparison to those mice treated with saline.

In homeopathic medicine, administration of highly dilute amounts of Thuja, is understood to cause a development of future immunity to antigens that cause increases in pro-inflammatory cytokines, which result in various inflammation related symptoms. One such symptom, a runny nose, is commonly treated with homeopathic amounts of Thuja.

In an embodiment of the present invention, which is set forth in greater detail in the examples below, the homeopathic composition comprises Thuja occidentalis. A serving of the homeopathic composition comprises from about 4× to about 8× of Thuja occidentalis. The preferred dosage of a serving of the homeopathic composition comprises about 6× of Thuja occidentalis.

In an embodiment of the present invention, which is set forth in greater detail an example below, the homeopathic composition comprises Thussilago petasites (butterbur), Allium cepa (red onion), Ambrosia artemisiifolia, Echineacea angustifolia, Euphrasia officinalis (eyebright), Pulsatilla nigricans, Pothos foetidus, Ignatia amara (St Ignatius' bean), and Thuja occidentalis (Tree of Life). The composition is provided in any acceptable and suitable oral dosage form as known in the art to alleviate the symptoms of an allergy.

The present invention is comprised of components that have been shown to elicit symptoms that are present in allergic response when administered to volunteers in large amounts. It is herein understood by the inventors that administration of such compounds in highly dilute forms, will act to induce the body of a mammal to produce immunogens able to alleviate the symptoms associated with allergies.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a powder or tablet suitable for dilution in water in accordance with the preparation and administration of homeopathic formulas.

Furthermore, the dosage form of the homeopathic supplement may be provided in accordance with customary processing techniques for herbal and nutritional supplements in any of the forms mentioned above. Additionally, the homeopathic supplement set forth in the example embodiment herein may contain any appropriate number and type of excipients, as is well known in the art.

The present homeopathic composition or those similarly envisioned by one of skill in the art may be utilized in methods to alleviate the symptoms of an allergy.

Although the following example illustrates the practice of the present invention in one of its embodiments, the example should not be construed as limiting the scope of the invention. Other embodiments will be apparent to one of skill in the art from consideration of the specifications and example.

### EXAMPLES

### Example 1

A homeopathic composition, for the alleviation of allergy symptoms, comprising the following ingredients, in homeopathically acceptable levels of dilution, per serving is prepared for consumption as a time release tablet to be consumed once daily:
Thussilago petasites (butterbur) providing 24 mg of petasins, Allium cepa (red onion), Ambrosia artemisiifolia, Echineacea angustifolia, Euphrasia officinalis (eyebright), Pulsatilla nigricans, Pothos foetidus, Ignatia amara (St Ignatius' bean), and Thuja occidentalis (Tree of Life)

### Example 2

A homeopathic composition, for the alleviation of allergy symptoms, comprising the following ingredients, in homeopathically acceptable levels of dilution, per serving is prepared for consumption as a tablet to be consumed three times daily:

Thussilago petasites (butterbur) providing 8 mg of petasins, Allium cepa (red onion), Ambrosia artemisiifolia, Echineacea angustifolia, Euphrasia officinalis (eyebright), Pulsatilla nigricans, Pothos foetidus, Ignatia amara (St Ignatius' bean), and Thuja occidentalis (Tree of Life)

Extensions and Alternatives

In the foregoing specification, the invention has been described with a specific embodiment thereof, however, it will be evident that various modifications and changes may be made thereto without departing from the broader spirit and scope of the invention.

What is claimed:

1. An orally administrable therapeutic composition comprising a effective amount of Thussilago petasites (butterbur) and a diluted amount of an immunogen; wherein said immunogen is Ambrosia artemisiifolia and has an immunotherapeutic effect against an allergen in a warm blood animal.

2. The composition of claim 1 wherein the Thussilago petasites (butterbur) acts to inhibit leukotriene biosynthesis.

3. The composition of claim 2 wherein the inhibition of leukotriene biosynthesis will effectively alleviate the symptoms of an allergy.

4. The composition of claim 1 wherein the Ambrosia artemisiifolia is present in homeopathically acceptable amounts.

5. The composition of claim 2 wherein the homeopathically acceptable amounts of Ambrosia artemisiifolia are able to induce the body of a mammal to produce immunogens able to alleviate the symptoms of an allergy.
